(19) United States
(12) Patent Application Publication
(10) Pub. No.: US 2012/0230974 A1
(43) Pub. Date: Sep. 13, 2012

(54) ALKALINE ALPHA GALACTOSIDASE FOR THE TREATMENT OF FABRY DISEASE

(75) Inventors: Yoseph Shaaltiel, Kibbutz HaSolelim (IL); Tehila Ben-Moshe, Koranit (IL); Yaniv Azulay, Akko (IL)

(73) Assignee: PROTALIX LTD, CARMIEL (IL)

(21) Appl. No.: 13/510,340

(22) PCT Filed: Nov. 17, 2010
(86) PCT No.: PCT/IL10/00956

§ 371 Date: May 17, 2012

Related U.S. Application Data
(60) Provisional application No. 61/261,787, filed on Nov. 17, 2009.

(51) Int. Cl. A61K 38/47

(52) U.S. Cl. 424/94.61

(57) ABSTRACT

A method of treating Fabry is provided. The method comprises administering to a subject in need thereof a therapeutically effective amount of alkaline alpha galactosidase, thereby treating Fabry disease.

### RELATED APPLICATION/S

This application claims the benefit of priority under 35 USC 119(e) of U.S. Provisional Patent Application No. 61/261,787 filed Nov. 17, 2009, the content of which is incorporated herein by reference in their entirety.

### FIELD AND BACKGROUND OF THE INVENTION

The present invention, in some embodiments thereof, relates to alkaline alpha galactosidase for the treatment of Fabry disease.

Fabry disease (also known as Fabry's disease, Anderson-Fabry disease, angiokeratoma corporis diffusum and alpha-galactosidase A deficiency) is a rare X-linked recessive (inherited) lysosomal storage disease.

The pathophysiology of the disease is a deficiency of the enzyme alpha galactosidase A (a-GAL A, encoded by GLA). A variety of mutations in the gene encoding the enzyme affect the synthesis, processing, and stability of this enzyme, causing its substrate, a glycolipid, known as globotriaosylceramide (abbreviated as Gb₃, GL-3, or ceramide trihexoside), to accumulate within the blood vessels, other tissues, and organs. This accumulation leads to an impairment of their proper function.

The condition affects hemizygous males (i.e. all males), as well as homozygous, and potentially heterozygous (carrier), females. Whilst males typically experience severe symptoms, women can range from being asymptomatic to having severe symptoms. This variability is thought to be due to X-inactivation patterns during embryonic development of the female.

Several lines of evidence suggest that enzyme replacement therapy (ERT) may be beneficial for patients with Fabry disease. For example, it has been demonstrated in cell cultures of fibroblasts obtained from patients with this disease that enzyme present in the culture medium is specifically transported to lysosomes. Clinical trials of enzyme replacement therapy have been reported for patients with Fabry disease using infusions of normal plasma (Mapes et al., 1970, Science 169: 987-989); .alpha.-galactosidase A purified from placenta (Brady et al., 1973, New Eng. J. Med. 279: 1163); or alpha.-galactosidase A purified from spleen or plasma (Desnick et al., 1979, Proc. Natl. Acad. Sci. USA 76: 5326-5330). In one study (Desnick et al.) intravenous injection of purified enzyme resulted in a transient reduction in the plasma levels of the substrate, globotriaosylceramide. However, due to the limited availability of the human enzyme obtained from human sources, insufficient quantities were available for further study.

Recombinant enzyme replacement therapies are available to functionally compensate for alpha-galactosidase deficiency. Agalsidase alpha (Shire PLC, Replagal™) and beta (Fabrazyme™, Genzyme) are both recombinant forms of the human α-galactosidase A enzyme and both have the same amino acid sequence as the native enzyme. Agalsidase alpha and beta differ in the structures of their oligosaccharide side chains. Both products have been proven efficacious in clinical studies with regard to clearance of Gb₃ from plasma, kidney cells (such as capillary endothelial cells, Glomerular endothelial cells, noncapillary endothelial cells and noncapillary smooth muscle cells), capillary endothelia cells of the cardiac and of the skin (Eng, Guffon et al. 2001; Germain, Waldek et al. 2007; Schaefer, Tylki-Szymanska et al. 2009).

Unfortunately it has become clear that clinical responses to ERT in Fabry patients are far less spectacular than those shown by Gaucher patients receiving a comparable intervention.

### RELATED ART

WO 04/096978; WO 98/13469; WO 08/132743. WO 08/075957

### SUMMARY OF THE INVENTION

According to an aspect of some embodiments of the present invention there is provided a method of treating Fabry, the method comprising administering to a subject in need thereof a therapeutically effective amount of alkaline alpha galactosidase, thereby treating Fabry disease.

According to an aspect of some embodiments of the present invention there is provided a pharmaceutical composition comprising as an active ingredient alkaline alpha galactosidase and a pharmaceutically acceptable carrier.

According to an aspect of some embodiments of the present invention there is provided a method of treating Fabry disease in a subject treated with acid alpha galactosidase, the method comprising administering to the subject a therapeutically effective amount of alkaline alpha galactosidase following the treatment with acid alpha galactosidase, thereby treating Fabry disease.

According to an aspect of some embodiments of the present invention there is provided an alkaline alpha galactosidase for use in the treatment of Fabry disease in a subject in need thereof.

According to some embodiments of the invention, the subject has been treated with acid alpha galactosidase.

According to some embodiments of the invention, the alkaline alpha galactosidase is a genetically modified human alpha galactosidase.

According to some embodiments of the invention, the alkaline alpha galactosidase is a plant alpha galactosidas.

According to some embodiments of the invention, the alkaline alpha galactosidase is a purified protein.

According to some embodiments of the invention, the alkaline alpha galactosidase is a recombinant protein.

According to some embodiments of the invention, the plant is a member of a plant family selected from the group consisting of Cucurbitaceae, Lamiaceae, Piperaceae, Solanaceae, Leguminosae, Cruciferae, Coffea and Gramineae family.

According to some embodiments of the invention, the alkaline alpha galactosidase is as set forth in SEQ ID NO: 2, 4, 5, 7, 9, 11, 13, 15, 17, 19 and 21.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. [IF IMAGES, REPHRASE] With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a calibration curve of N-Dodecanoyl-NBD-ceramide trihexoside (NBD-Gb₃) on HP-TLC (silica gel-60 plate; Chloroform: Methanol: H₂O [100:42:6] as mobile phase). Lanes show increasing amounts (ng) of NBD-Gb₃

FIG. 2 shows hydrolysis of Gb₃-NBD (lower spot) to lactosylceramide-NBD (upper spot) by plant recombinant human alpha gal (citrate phosphate buffer, pH 4.6), as observed by HP-TLC. Left lane: prh alpha Gal catalyzed reaction; middle lane: uncatalyzed reaction mixture; right lane: Gb₃-NBD standard.

FIG. 3 shows hydrolysis of Gb₃-NBD (lower spot) to lactosylceramide-NBD (upper spot) by Replagal™, prh-alpha-Gal and GCB-a-Gal (endogenous green coffee bean) in citrate phosphate buffer, pH 4.6 (lanes 1-2) and phosphate buffer, pH 6.5 (lanes 3-5).

FIG. 4 shows hydrolysis of Gb₃-NBD (lower spot) to lactosylceramide-NBD (upper spot) by prh-alpha-Gal (lane 1) and GCB-a-Gal (endogenous green coffee bean; lane 2) in PBS, pH 7.4. Lane 3- Gb₃-NBD standard.

FIG. 5 shows GB₃-NBD levels in plasma of WT and Fabry mice (measured by fluorescence) one hour (1 h) and 24 hours (24 h) following injection of GB₃-NBD.

FIG. 6 shows GB₃-NBD levels in liver of WT and Fabry mice (measured by fluorescence) one hour (1 h) and 24 hours (24 h) following injection of GB₃-NBD.

### DESCRIPTION OF SPECIFIC EMBODIMENTS OF THE INVENTION

The present invention, in some embodiments thereof, relates to alkaline alpha galactosidase for the treatment of Fabry disease.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Fabry disease is a rare X-linked recessive (inherited) lysosomal storage disease, which can cause a wide range of systemic symptoms. A deficiency of the enzyme alpha galactosidase A due to mutation causes a glycolipid known as globotriaosylceramide (abbreviated as Gb₃, GL-3, or ceramide trihexoside) to accumulate within the blood vessels, other tissues, and organs. This accumulation leads to an impairment of their proper function.

Recombinant human alpha-GAL-A has the ability to restore enzyme function in patients, and currently two ERTs using this enzyme are commercially available; agalsidase-alpha (Replagal™, Shire PLC) that was approved in Europe and agalsidase-beta (Fabrazyme™, Genzyme) that was approved both in Europe and in the United States. These enzymes are difficult to manufacture and as such are expensive. Recently, contamination at Genzyme's Allston, Mass. plant caused a worldwide shortage of Fabrazyme, and supplies were rationed to patients at one-third the recommended dose.

Since the natural site of the enzymes is the lysosome (i.e. organelles characterized by a low pH), alpha galactosidases exert their maximal activity at these low pH levels, whilst their activity at higher pH levels is compromised and considered negligible. Thus, for example, α-galactosidase used in ERT is unable to hydrolyze terminal galactosylated glycolipids in the serum of Fabry patients.

Therefore, the present inventors now suggest treating Fabry disease using alpha galactosidase that is selected active in the serum. The use of serum active enzyme is advantageous compared to lysosomal active enzyme mainly because of the potential to increase efflux of Gb₃ from the cells. In addition, such a serum active form of the enzyme would be efficient in removing and preventing glycosphinglipids deposit within blood vessel walls which promote inflammation [Bodary et al., TCM 17(4):129-133]. For example, in Fabry disease, the major pathogenesis results from the accumulation of Gb₃ in the vascular endothelium, leading to vascular occlusion of small vessels, ischemia and infarction of these vessels and ischemia and infarction of the kidney, heart and brain [Desnick et al., 2003, Annals of Internal Medicine, 138(4):338-346]. Finally, by negating the need for lysosomal trafficking, ERT becomes much more accessible since robust, cost-effective host systems e.g., plants, can be employed.

Thus, according to an aspect of the present invention there is provided a method of treating Fabry disease, the method comprising, administering to a subject in need thereof a therapeutically effective amount of alkaline alpha galactosidase, thereby treating Fabry disease.

As used herein “alpha galactosidase” refers to E.C. 3.2.1.22.

As used herein “alpha galactosidase” refers to alpha galactosidase A or B.

As used herein the phrase “alkaline-α-galactosidase activity” refers to the ability of the enzyme to optimally hydrolyse terminal-linked α-galactose moieties from galactose-containing oligosaccharides under neutral to basic pH conditions (e.g., about pH 7-7.5). Normal serum pH is slightly alkaline and ranges from about 7.35-7.45.

It will be appreciated that the alkaline alpha galactosidase of some embodiments of the invention may be optimally active under neutral to basic pH conditions but may still display activity under acidic pH conditions (i.e., of the lysosome i.e., 4.5 or above that of the lysosome).

In a specific embodiment the enzyme is active under acidic to basic pH conditions (i.e., about pH 4.2-7.5 or 4.5-7.5).

In another specific embodiment the enzyme is active under basic pH conditions (e.g., about 7.35-7.5).

In yet another specific embodiment the enzyme is active under pH of about 6.5-7.5.

As used herein “acid-α-galactosidase” refers to the ability of an enzyme to optimally hydrolyse terminal-linked α-galactose moieties from galactose-containing oligosaccharides under acidic pH conditions (e.g., about pH 4.2-4.5 or 4.0-5.0).

The alkaline alpha galactosidase enzyme of the invention can be of any human, animal or plant source, provided no adverse immunological reaction is induced upon in vivo administration (e.g., plant to human).

To reduce immunological reaction, a non-human preparation (e.g., of plant alkaline alpha galactosidase) can be co-administered with the human enzyme (i.e., acid human alpha galactosidase).

Human alpha galactosidase is commercially available [agalsidase alpha Replagal®, Shire or agalsidase beta Fabrazyme®, Genzyme).

The alkaline alpha galactosidase enzyme of the invention can be purified (e.g., from plants) or generated by recombinant DNA technology.

Specific examples of alkaline alpha galactosidases which can be used in accordance with the present teachings are provided in US Patent Application 20070036883, WO03/097791 each of which is hereby incorporated by reference in its entirety.

Thus, alkaline alpha galactosidase can be a member of the plant family selected from the group consisting of Cucurbitaceae, Lamiaceae, Piperaceae, Solanaceae, Leguminosae, Cruciferae and Gramineae family.

According to a specific embodiment, the alkaline alpha galactosidase is from melon.

P.-R. Gaudreault and J. A. Webb have described in several publications, (such as “Alkaline alpha-galactosidase in leaves of Cucurbita pepo”, Plant Sci. Lett. 24, 281-288, 1982, “Partial purification and properties of an alkaline alpha-galactosidase from mature leaves of Cucurbita pepo”, Plant Physiol., 71, 662-668, 1983, and ‘Alkaline alpha-galactosidase activity and galactose metabolism in the family Cucurbitaceae”, Plant Science, 45, 71-75, 1986), a novel alpha-galactosidase purified from young leaves of Cucurbita pepo, that has an optimal activity at alkaline conditions (pH 7.5).

Alpha-galactosidase activity at alkaline pH has been observed in other cucurbit tissue, such as cucumber fruit pedicels, young squash fruit and young melon fruit (“Melons: Biochemical and Physiological Control of Sugar Accumulation, In: Encyclopedia of Agricultural Science, vol. 3, pp. 25-37, Arntzen, C. J., et al., eds. Academic Press, New York, 1994).

Bachmann et al. (“Metabolism of the raffinose family oligosaccharides in leaves of Ajuga reptens L.”, Plant Physiology 105:1335-1345, 1994) reports that Ajuga reptens plants (common bugle), a stachyose translocator from the unrelated Lamiaceae family also contains an alkaline alpha-galactosidase. This enzyme was partially characterized and found to have high affinity to stachyose. Also, leaves of the Peperomia camptotricha L. plant, from the family Piperaceae, show alpha-galactosidase activity at alkaline pH, suggesting that they also contain an alkaline alpha-galactosidase enzyme (Madore, M., “Catabolism of raffinose family oligosaccharides by vegetative sink tissues”, In: Carbon Partitioning and Source-Sink Interactions in Plants, Madore, M. and Lucas, W. J. (eds.) pp. 204-214, 1995, American Society of Plant Physiologists, Maryland). Similarly, Gao and Schaffer (Plant Physiol. 1999; 119:979-88, which is incorporated fully herein by reference) have reported an alpha galactosidase activity with alkaline pH optimum in crude extracts of tissues from a variety of species including members of the Cucurbit and Coleus (Lamiaceae) families.

Specific examples of plant alkaline alpha galactosidase sequences are provided in SEQ ID NOs: 1-4 and 19-20 (C. melo), 5-6 (T. tetragonioides), 7-8 and 17-18 (C. sativus), 9-12 (Zea mays), 13-14 (Oruza sativa), 15-16 (Pisum sativum) and 21 (Coffea Arabica).

Other examples are provided in the Examples section which follows.

The enzyme may act in the serum alone (upon in vivo administration) and optionally in the cells (e.g., cytoplasm and/or lysosome). In a specific embodiment the enzyme is active also in the lysosome. In the latter configuration the enzyme is characterized by a phosphorylated high mannose for incorporation into cells. PCT WO2008/132743 teaches recombinant plant-produced alpha galactosidase which can be incorporated into lysosomes.

WO2009/024977 teaches methods of conjugating M6P to alpha galactosidase for improved uptake into the lysosomes using M6P-PEG₁₂-COOH or M6P-PEG₈-maleimide. Each of the above references is hereby incorporated herein.

Alpha-galactosidase (e.g., human) can be artificially modified to act under neutral to basic pH conditions (e.g., pH 7-10).

Methods of generating enzymes with improved catalytic activity under alkaline pH conditions include directed evolution.

As used herein the phrase “in vitro evolution process” or “a directed evolution process” refers to the manipulation of genes and selection or screening of a desired activity. A number of methods, which can be utilized to effect in vitro evolution, are known in the art.

Chen et al. 2003 Chemistry and Biology 15:1277-1286 and Maranville 2000 Eur. J. Biochem. 267:1495-1501 (each of which is herein incorporated by reference in its entirety) describe modifications of alpha galactosidase for gaining activity in said neutral to basic pH range.

General outline of directed evolution is provided in Tracewell C A, Arnold F H “Directed enzyme evolution: climbing fitness peaks one amino acid at a time” Curr Opin Chem Biol. 2009 February;13(1):3-9. Epub 2009 Feb. 25; Gerlt J A, Babbitt P C, Curr Opin Chem Biol. 2009 February;13(1):10-8. Epub 2009 Feb. 23 (either of which is hereby incorporated by reference in its entirety).

Nucleic acid sequences used for producing the enzymes by recombinant means may be complementary polynucleotide sequences, genomic sequences or composite sequences. The polynucleotides may also be codon optimized according to the host system used.

As used herein the phrase “complementary polynucleotide sequence” refers to sequences, which originally result from reverse transcription of messenger RNA using a reverse transcriptase or any other RNA dependent DNA polymerase. Such sequences can be subsequently amplified in vivo or in vitro using a DNA dependent DNA polymerase.

As used herein the phrase “genomic polynucleotide sequence” refers to sequences, which are derived from a chromosome and thus reflect a contiguous portion of a chromosome.

As used herein the phrase “composite polynucleotide sequence” refers to sequences, which are at least partially complementary and at least partially genomic. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements.

As mentioned, the enzymes of the present invention can be produced by recombinant DNA techniques.

Thus, there is provided a method of producing a recombinant alkaline α-galactosidase protein. The method is effected by several method steps, in which in a first step an expression construct, which includes any of the polynucleotides of the present invention positioned under the transcriptional control of a regulatory element, such as a promoter, is introduced into a cell.

In the next method step transformed cells are cultured under effective conditions, which allow the expression of the polypeptide encoded by the polynucleotide.

It will be appreciated that the enzyme need not be recovered from the host cell (e.g., plant cell). In fact the present invention also contemplates treatment with plant cells expressing the alkaline alpha galactosidase e.g., such as for oral administration.

However, according to an alternative embodiment, the enzyme is recovered from the host cell, and purification is effected according to the end use of the recombinant polypeptide. For clinical applications the enzymes are purified sterile and to clinical grade.

Depending on the host/vector system utilized, any of a number of suitable transcription and translation elements including constitutive and inducible promoters, transcription enhancer elements, transcription terminators, and the like, can be used in the expression vector [see, e.g., Bitter et al., (1987) Methods in Enzymol. 153:516-544].

Other than containing the necessary elements for the transcription and translation of the inserted coding sequence, the expression construct of the present invention can also include sequences engineered to enhance stability, production, purification, yield or toxicity of the expressed polypeptide. For example, the expression of a fusion protein or a cleavable fusion protein comprising the alkaline α-galactosidase and a heterologous protein can be engineered. Such a fusion protein can be designed so that the fusion protein can be readily isolated by affinity chromatography; e.g., by immobilization on a column specific for the heterologous protein. Where a cleavage site is engineered between the alkaline α-galactosidase moiety and the heterologous protein, the alkaline α-galactosidase protein can be released from the chromatographic column by treatment with an appropriate enzyme or agent that disrupts the cleavage site [e.g., see Booth et al. (1988) Immunol. Lett. 19:65-70; and Gardella et al., (1990) J. Biol. Chem. 265:15854-15859].

A variety of eukaryotic cells (e.g., mammalian or plant cells) can be used as host-expression systems to express the alkaline α-galactosidase coding sequence.

In cases where plant expression vectors are used, the expression of the alkaline α-galactosidase coding sequence can be driven by a number of promoters. For example, viral promoters such as the 35S RNA and 19S RNA promoters of CaMV [Brisson et al. (1984) Nature 310:511-514], or the coat protein promoter to TMV [Takamatsu et al. (1987) EMBO J. 6:307-311] can be used. Alternatively, plant promoters such as the small subunit of RUBISCO [Coruzzi et al. (1984) EMBO J. 3:1671-1680 and Brogli et al., (1984) Science 224:838-843] or heat shock promoters, e.g., soybean hsp17.5-E or hsp17.3-B [Gurley et al. (1986) Mol. Cell. Biol. 6:559-565] can be used. These constructs can be introduced into plant cells using Ti plasmid, Ri plasmid, plant viral vectors, direct DNA transformation, microinjection, electroporation and other techniques well known to the skilled artisan. See, for example, Weissbach & Weissbach, 1988, Methods for Plant Molecular Biology, Academic Press, NY, Section VIII, pp 421-463.

Other expression systems such as insects and mammalian host cell systems, which are well known in the art can also be used by the present invention.

In any case, alkaline α-galactosidase transformed cells are cultured under effective conditions, which allow for the expression of high amounts of recombinant alkaline α-galactosidase. Effective culture conditions include, but are not limited to, effective media, bioreactor, temperature, pH and oxygen conditions that permit protein production. An effective medium refers to any medium in which a cell is cultured to produce the recombinant alkaline α-galactosidase protein of the present invention. Such a medium typically includes an aqueous solution having assimilable carbon, nitrogen and phosphate sources, and appropriate salts, minerals, metals and other nutrients, such as vitamins. Cells of the present invention can be cultured in bioreactors, shake flasks. Culturing can be carried out at a temperature, pH and oxygen content appropriate for a recombinant cell. Such culturing conditions are within the expertise of one of ordinary skill in the art.

Depending on the vector and host system used for production, resultant proteins of the present invention may either remain within the recombinant cell (e.g., as described in WO2008/132743); or be secreted into the fermentation medium.

Following a certain time in culture, recovery of the recombinant protein is effected. The phrase “recovering the recombinant protein refers to collecting the fractions containing the recombinant protein (e.g., whole fermentation medium or cells) containing the protein and need not imply additional steps of separation or purification. Proteins of the present invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

Proteins of the present invention are preferably retrieved in “substantially pure” form. As used herein, “substantially pure” refers to a purity that allows for the effective use of the protein in the clinical applications (i.e., over 95%, 96%, 97%, 98%, 99%, or more free of contaminants.

As mentioned, aside from recombinant DNA technology, the enzyme can be purified from eukaryotic or prokaryotic systems naturally expressing same (i.e., no heterologous gene expression).

Methods of purifying alkaline alpha galactosidase from plants are well known in the art. See e.g., U.S. Pat. No. 6,607,901 which is hereby incorporated by reference teaches various methods for purification of alkaline alpha-galactosidase.

Alkaline alpha galactosidase produced according to the present teachings can be used for decreasing at least the plasma (serum) concentration of glycosphingolipids, particularly globotriaosylceramide [also abbreviated as Gb₃, GL-3 or ceramide trihexoside (CTH)].

Thus the present invention further provides for a method of treating Fabry disease. The method comprising administering to a subject in need thereof a therapeutically effective amount of alkaline alpha galactosidase, thereby treating Fabry disease.

It will be appreciated that alkaline alpha galactosidase of the present teachings can also be used as adjuvant therapy for complementing treatment with the typically used (acid) alpha galactosidase. In this case a therapeutically effective amount of the basic enzyme is administered following treatment with the acid enzyme (e.g., Fabrazyme®, Genzyme, Cambridge, Mass.) such as for reducing substrate accumulation in organs such as the kidney.

The subject is one that has been diagnosed with Fabry disease. The subject may be treated according to the present teachings from early onset to later stages of the disease.

According to a specific embodiment, the subject is treated already at early stages of the disease to prevent slow accumulation of the substrate.

Therapeutic efficacy as well as treatment regimen can be determined also by determining the levels of serum substrate such as described in WO 08/075957 which is hereby incorporated by reference in its entirety as well as in the Examples section which follows.

The alkaline alpha galactosidase (alone or in combination with alpha galactosidase (e.g., active in acidic pH) or cells expressing same as described hereinabove can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to at least the alkaline alpha galactosidase accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, inrtaperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

The term “tissue” refers to part of an organism consisting of an aggregate of cells having a similar structure and/or a common function. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuos infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients effective to prevent, alleviate or ameliorate symptoms of a disorder or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide tissue levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### EXAMPLE 1

### Calibration Curve of N-Dodecanoyl-NBD-Ceramide Trihexoside (NBD-Gb₃) on HP-TLC

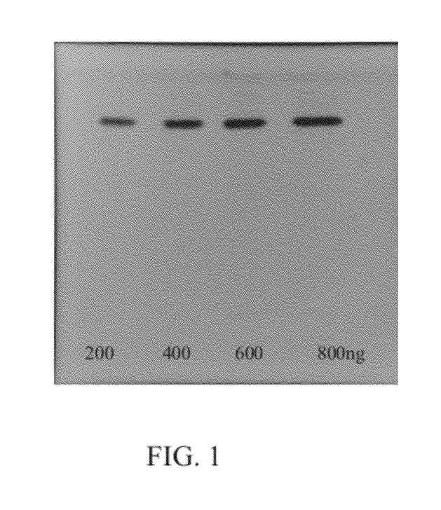

A stock solution of Dodecanoyl-NBD-ceramide trihexoside (NBD-Gb₃) 1 ug/ul [in ethanol] was diluted ten-fold to 0.1 ug/ul and was loaded on a HP-TLC silica-60 plate. Chloroform: Methanol: H₂O [100:42:6] was used as mobile phase (FIG. 1).

### EXAMPLE 2

### In vitro Hydrolysis of Gb₃-NBD by Plant Recombinant Human (prh)-alpha-Gal in pH 4.6

Methods of expressing prh-alpha Gal are described in WO2008/132743 (Alpha1-KDEL).

The following protocol was used.

90 μl activity buffer [citrate phosphate pH=4.6]

5 μl of NBD-Gb₃ 1 ug/ul [50 ug/ml in reaction]

5 ul of Plant a-Gal 5 mg/ml [0.05 mg/ml in reaction]

Incubation for 60 min in 37° C.

Lipid extraction:

100 ul chloroform were added followed by vortex

50 ul methanol were added followed by vortex

lower phase separation

Injection on TLC silica-60 plate, Chloroform: Methanol: H₂O [100:42:6].

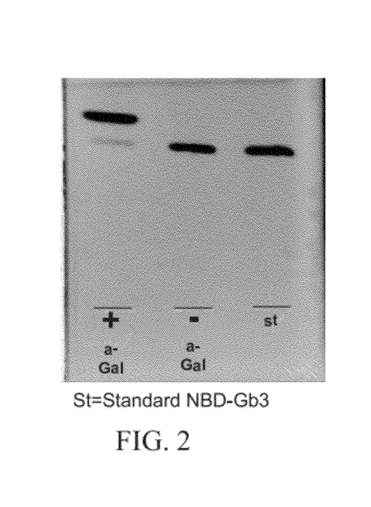

As can be seen from FIG. 2, incubation with the enzyme caused substrate hydrolysis and upshift of the band corresponding to NBD-GB₃ to its product, NBD-lactosylceramide (NBD-Gb₂).

Conclusion:

Almost all NBD-Gb₃ was hydrolyzed to NBD-lactosylceramide (NBD-Gb₂).

### EXAMPLE 3

In vitro Hydrolysis of Gb₃-NBD by Alpha Galactosidases from Different Sources under Various pH Conditions

Enzymes:

Green Coffee Bean-GCB a-Gal (Sigma #G8507)

Plant recombinant human alpha galactosidase was produced as described in WO2008/132743 (alpha-Gal-KDEL).

Commercial recombinant human alpha galactosidase (Replagal, Shire)

The following protocol was used for NBD-Gb₃ hydrolysis:

90 μl activity buffer [citrate phosphate pH=4.6; phosphate buffer pH=6.5

10 μl of NBD-Gb₃ 0.35 ug/ul in Ethanol [35 ug/ml in reaction]

5 μl of a-Gal 1 mg/ml [50 ug/ml in reaction]

Incubation for 60 min in 37° C.

100 μl chloroform were added followed by vortex

50 μl methanol were added followed by vortex

lower phase separation

Injection on TLC silica-60 plate, Chloroform: methanol: H₂O [100:42:6] as mobile phase.

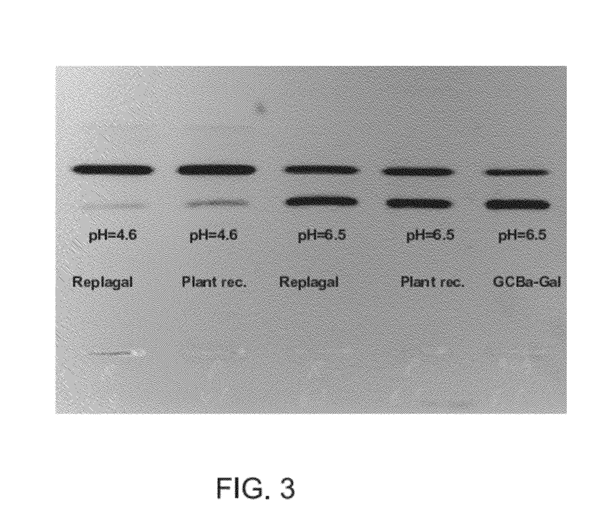

FIG. 3 shows the hydrolysis of Gb₃-NBD (lower spot) to lactosylceramide-NBD (upper spot) by Replagal, prh-alpha-Gal and GCB-a-Gal (endogenous green coffee bean) under various pH conditions.

Conclusions:

Both Replagal and prh-alpha-Gal can partially hydrolyze Gb₃ in pH 6.5.

Green coffee bean alpha gal can hydrolyze NBD-Gb₃ even at these acidic conditions.

### EXAMPLE 4

### Activity Assay with NBD-Gb₃ in PBS [pH=7.4]

a-Gals: prh-alpha galactosidase, GCB a-Gal (endogenous green coffee bean).

80 μl PBS (sigma), pH=7.4

10 μl of NBD-Gb₃ 0.1 μg/ul in Ethanol [10 ug/ml in reaction]

10 μl of a-Gal 1 mg/ml [100 μg/ml in reaction]

Incubated for 60 min in 37° C.

150 μl chloroform: methanol (2:1) were added followed by vortex

the lower phase pulled out

speed-vac was effected to complete evaporation and the pellet dissolved in 50 μl Chloroform: methanol [1:1]

All samples were injected on HP-TLC silica-60 plate [40 ul ]

Chloroform: methanol: H₂O [100:42:6].

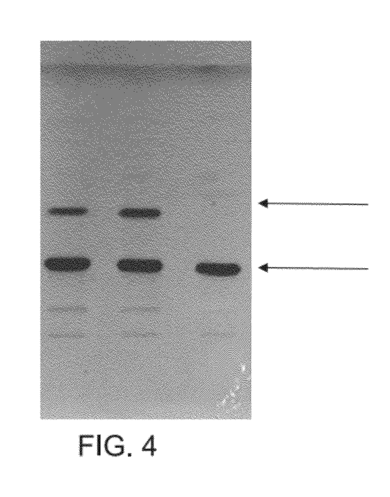

FIG. 4 shows GB₃-NBD (bottom arrow) and lactosylceramide-NBD product (top arrow) following incubation with alphagalactosidase in pH 7.4.

From left to right:

Lane 1: Plant recombinant human (prh) alpha galactosidase.

Lane 2: endogenous Green coffee been alpha galactosidase.

Lane 3 : no enzyme.

Conclusion: Both tested enzymes Green coffee bean alpha Gal and plant recombinant human alpha Gal demonstrate enzymatic activity under neutral to basic pH. However clearly the Green Coffee Bean alpha galactosidase works better as can be seen by the upshift. The Green Coffee Bean alpha galactosidase is more active under alkaline pH conditions when compared to the acidic conditions shown in FIG. 3.

### EXAMPLE 5

### Biodistribution of Gb₃

For testing the hypothesis that circulating Gb₃ can reach and accumulate in organs, organ uptake and biodistribution of fluorescent Gb₃ (N-Dodecanoyl-NBD-ceramide trihexoside) in wild type (WT) and Fabry mice were effected.

Test System:

Animals: Mice: Fabry mice and WT mice

Group Size: total 12 male mice, n=2

TABLE 1

| | | No of | | | |
|---|---|---|---|---|---|
| Group | Test item | mice | Harvest | Dose | Route |
| 1 | N-Dodecanoyl- | 2 wt | 1 hr after dosing | 320 ug/kg | iv |
| 2 | NBD-ceramide | 2 wt | 24 hr after dosing | | |
| 3 | trihexoside | 2 Fabry | 1 hr after dosing | | |
| 4 | | 2 Fabry | 24 hr after dosing | | |
| 5 | Saline | 2 wt | 24 hr after dosing | — | |
| 6 | Saline | 2 Fabry | 24 hr after dosing | — | |

Materials: fluorescent Gb₃ (N-Dodecanoyl-NBD-ceramide trihexoside; Catalog #: 1631, Matreya Pa. 16823 USA

α-Galactosidase-A-deficient mice:

Jackson B6J129Gla α-galactosidase-A-deficient mice (“Fabry mice”) were purchased from Jackson Laboratories. These mice are characterized by being totally deficient in α-Galactosidase-A activity and progressively accumulate Gb3 in both plasma and in the lysosomes of most tissues (in particular, the liver, spleen, heart, skin, and kidneys). In addition, these mice have no clinical disease phenotype and survive a normal laboratory life span (>2 years). Hemizygous affected males were bred to homozygous affected females, thereby providing only affected offspring. For these studies, all mice were affected adult males 12 to 30 weeks of age at study initiation.

α-Galactosidase-A assay:

The level of active α-galactosidase A was determined against a calibration curve of the activity of a commercial α-galactosidase (Fabrazyme®, Genzyme, Cambridge, Mass.) plotted for the concentration range of 200-12.5 ng/ml. Activity was determined using p-nitrophenyl-α-D-galactopyranoside (Sigma) as a hydrolysis substrate. The assay buffer contained 20 mM citric acid, 30 mM sodium phosphate, 0.1% BSA and 0.67% ethanol at pH 4.6. The assay was performed in 96 well ELISA plates (Greiner # 655061). 50 μl of tissue sample lysates were incubated with 150 μl assay buffer and 30 μl substrate was added to obtain a final concentration of 8 mM. The reaction mixture was incubated at 37° C. for 90 minutes and results were plotted against the calibration results. Product (p-nitrophenyl; PNP) formation was detected by absorbance at 405 nm. Absorbance at 405 nm was measured before initiating the reaction. After 90 minutes, 100 μl of 1.98 M sodium carbonate was added to each well in order to terminate the reaction, and absorbance at 405 nm was measured again.

Administration: IV injection, tail vein.

Plasma:

Gb₃-NBD was injected to wild type and Fabry mice. Blood was collected 1 hour and 24 hours following injection and plasma was prepared using accepted methods. Gb₃-NBD levels were determined using Fluorescent Elisa reader (Infinite M200; Tecan, Switzerland), subtracting basal fluorescent levels plasma of control mice injected with saline.

Gb₃ presence was also detected with HP TLC (CAMAG, Switzerland)

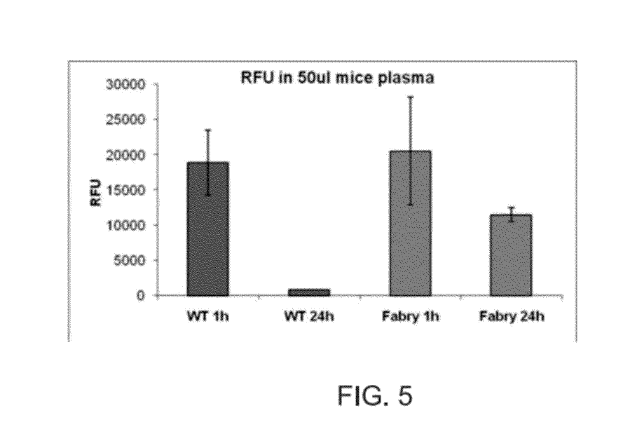

Results of Fluorescent Gb₃ in plasma of WT and Fabry mice are shown in FIG. 5. Gb₃-NBD levels in plasma (FIG. 5) as detected by fluorescence levels also showed similar fluorescence in WT and Fabry mice 1 hour following injections, while plasma from mice 24 hours following injection, showed fluorescence only in Fabry mice.

Results show that Gb₃-NBD accumulates in plasma of Fabry mice, while its absence in WT mice could indicate it is hydrolyzed by the endogenous alpha Gal enzyme.

Gb₃-NBD levels in organs of mice injected with Gb3-NBD.

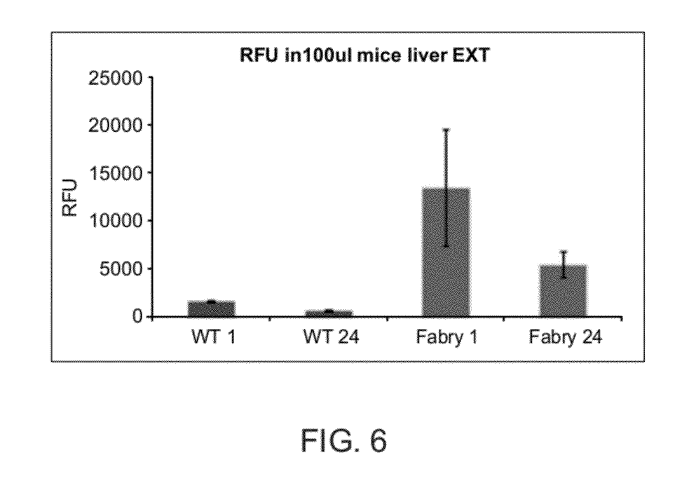

Organs (liver, kidney heart and spleen) were collected from the mice of the experiment described above, and Gb₃-NBD levels were determined by fluorescence detector and HPTLC as described. As the experiment was initial, low levels of Injected Gb₃ NBD were given. Fluorecent levels were only detectable in Plasma (FIG. 5) and Liver (FIG. 6).

Results from HPTLC showed bands that could be identified as Gb₃-NBD only in Fabry mice, however, results were inconclusive due to high background (results not shown).

It is believed that if higher levels of substrate were injected, in numerous injections, accumulation could be detected in Fabry mice in other organs, e.g. Kidney hart and spleen, in a similar manner to the liver

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of treating Fabry disease, the method comprising administering to a subject in need thereof a therapeutically effective amount of alkaline alpha galactosidase, thereby treating Fabry disease.

2. A pharmaceutical composition comprising as an active ingredient alkaline alpha galactosidase and a pharmaceutically acceptable carrier.

3. A method of treating Fabry disease in a subject treated with acid alpha galactosidase, the method comprising administering to the subject a therapeutically effective amount of alkaline alpha galactosidase following said treatment with acid alpha galactosidase, thereby treating Fabry disease.

4-5. (canceled)

6. The method of claim 1, wherein said alkaline alpha galactosidase is a genetically modified human alpha galactosidase.

7. The method of claim 1, wherein said alkaline alpha galactosidase is a plant alpha galactosidase.

8. The method of claim 1, wherein said alkaline alpha galactosidase is a purified protein.

9. The method of claim 1, wherein said alkaline alpha galactosidase is a recombinant protein.

10. The method of claim 7, wherein the plant is a member of a plant family selected from the group consisting of Cucurbitaceae, Lamiaceae, Piperaceae, Solanaceae, Leguminosae, Cruciferae and Gramineae family.

11. The method of claim 1, wherein said alkaline alpha galactosidase is selected from the group consisting of SEQ ID NO: 2, 4, 5, 7, 9, 11, 13, 15, 17 and 19.

12. The pharmaceutical composition of claim 2, wherein said alkaline alpha galactosidase is a genetically modified human alpha galactosidase.

13. The method of claim 3, wherein said alkaline alpha galactosidase is a genetically modified human alpha galactosidase.

14. The pharmaceutical composition of claim 2, wherein said alkaline alpha galactosidase is a plant alpha galactosidase.

15. The method of claim 3, wherein said alkaline alpha galactosidase is a plant alpha galactosidase.

16. The pharmaceutical composition of claim 2, wherein said alkaline alpha galactosidase is a purified protein.

17. The method of claim 3, wherein said alkaline alpha galactosidase is a purified protein.

18. The pharmaceutical composition of claim 2, wherein said alkaline alpha galactosidase is a recombinant protein.

19. The method of claim 3, wherein said alkaline alpha galactosidase is a recombinant protein.

20. The pharmaceutical composition of claim 14, wherein the plant is a member of a plant family selected from the group consisting of Cucurbitaceae, Lamiaceae, Piperaceae, Solanaceae, Leguminosae, Cruciferae and Gramineae family.

21. The method of claim 15, wherein said the plant is a member of a plant family selected from the group consisting of Cucurbitaceae, Lamiaceae, Piperaceae, Solanaceae, Leguminosae, Cruciferae and Gramineae family.

22. The pharmaceutical composition of claim 2, wherein said alkaline alpha galactosidase is selected from the group consisting of SEQ ID NO: 2, 4, 5, 7, 9, 11, 13, 15, 17 and 19.

23. The method of claim 3, wherein said alkaline alpha galactosidase is selected from the group consisting of SEQ ID NO: 2, 4, 5, 7, 9, 11, 13, 15, 17 and 19.


SEQUENCE LISTING (21 sequences)

SEQ ID NO: 1 - DNA, 2259 bp, Cucumis melo
atgacggttggtgctggaattactatctccgatgcgaatttgacggtgttgggaaatcgtgttttatccgatgttcataataacattactctcacggcggcgccgggtggtggtgtgatgaacggcgccttcataggagttcaatctgatcagatcggtagtcgccgagtttttcctattgggaaattgatagggttgagattcttatgtgcttttcgattcaaattatggtggatgactcaaagaatggggtgttccggtcaagaagttccattcgagacacaatttcttgtggtggaaacacgtgatggttctaacattgccggaaatggagaggaaggcgatgccgtttatactgtttttcttcctattcttgaaggcgatttcagagctgttcttcaagggaatgataataatgaaattgaaatctgtttagaaagtggagatccaagtgtagatgggtttgagggtagccatttggtgtttgtgggtgctggatcagatccttttgaaaccattacttatgcagtcaagtctgttgaaaagcatttgcaaacttttgctcatcgcgaaagaaagaagatgcctgatattttgaactggttcggctggtgcacatgggatgctttctacactgatgtcacttcagatggcgtcaagaagggtcttgaaagctttgagaatggaggaattcctcccaagtttgtcattatcgatgatggatggcaatcagttgccaaggatgctactagtgctgattgcaaagctgataacacagcaaactttgcaaacaggttaactcacataaaagagaattacaaatttcaaaaagatggcaaagagggtgaaagaattgagaaccctgcactgggtcttcaacatattgtgtcctacatgaaagagaagcatgcgaccaagtatgtttatgtttggcatgccataacaggctactggggtggtgtgagtgctggagttaaagagatggaacaatatgagtccaagattgcgtacccggttgcatctcctggggtcgaatcaaatgagccatgtgatgctttgaatagcatcaccaaaactggacttggccttgtgaaccctgaaaaggttttcaacttctacaatgaacaacactcgtatcttgcgtctgctggtgttgatggagttaaagttgatgttcaaaacattcttgagacgcttggagcaggtcatggtggaagagttaaacttgctagaaaataccatcaggctcttgaggcatcgatttcccgaaactttcaagataacggaatcatttcgtgtatgagtcataataccgatggtttatacagttcaaagagaaatgctgttattcgagcatcggatgatttttggcctagagatccagcatctcacacgattcatatagcatcagttgcttacaactccttatttcttggggagtttatgcagccagattgggatatgtttcatagtcttcatcctatggccgaatatcacggagcagctcgtgccgtgggaggatgtgctatatatgtcagtgacaagcctggtcaacatgacttcaatcttttgaagaagcttgtcctccctgatggttctattctgagagctaagctccccggacggccgacaaaggactgcctttttacggatcctgctagagatggaaaaagtctattgaagatttggaatttgaatgatctatctggagttgttggggtctttaactgccaaggagcaggatggtgtaaggttggaaagaaaaacctcattcacgacgagaatccagacacgatcacgggggttattcgagcaaaagatgttagttatctatggaagattgcaggcgagtcctggacaggggatgcagtgatattctcccatcttgctggagaagttgtttacctgccacaagatgcatcgatgccaataaccttgaagcctcgagagttcgacgtcttcacggttgttcctgtcaaggaactagttaatgacatcaagtttgctcctataggtttgatcaagatgttcaactctggaggagcagtgaaagaaatgaaccatcaacctggaagttcgaatgtgtcgctgaaagttcggggttctgggccattcggggcatattcctcgagcaaaccgaagcgtgtagcagtcgactcggaggaggtagagttcatgtatgatgagggtggtttaatcaccattgacttgaaggtaccagagaaagagttgtacctttgggatataagaattgaacta

SEQ ID NO: 2 - protein, 753 aa, Cucumis melo
MTVGAGITISDANLTVLGNRVLSDVHNNITLTAAPGGGVMNGAFIGVQSDQIGSRRVFPIGKLIGLRFLCAFRFKLWWMTQRMGCSGQEVPFETQFLVVETRDGSNIAGNGEEGDAVYTVFLPILEGDFRAVLQGNDNNEIEICLESGDPSVDGFEGSHLVFVGAGSDPFETITYAVKSVEKHLQTFAHRERKKMPDILNWFGWCTWDAFYTDVTSDGVKKGLESFENGGIPPKFVIIDDGWQSVAKDATSADCKADNTANFANRLTHIKENYKFQKDGKEGERIENPALGLQHIVSYMKEKHATKYVYVWHAITGYWGGVSAGVKEMEQYESKIAYPVASPGVESNEPCDALNSITKTGLGLVNPEKVFNFYNEQHSYLASAGVDGVKVDVQNILETLGAGHGGRVKLARKYHQALEASISRNFQDNGIISCMSHNTDGLYSSKRNAVIRASDDFWPRDPASHTIHIASVAYNSLFLGEFMQPDWDMFHSLHPMAEYHGAARAVGGCAIYVSDKPGQHDFNLLKKLVLPDGSILRAKLPGRPTKDCLFTDPARDGKSLLKIWNLNDLSGVVGVFNCQGAGWCKVGKKNLIHDENPDTITGVIRAKDVSYLWKIAGESWTGDAVIFSHLAGEVVYLPQDASMPITLKPREFDVFTVVPVKELVNDIKFAPIGLIKMFNSGGAVKEMNHQPGSSNVSLKVRGSGPFGAYSSSKPKRVAVDSEEVEFMYDEGGLITIDLKVPEKELYLWDIRIEL

SEQ ID NO: 3 - DNA, 2316 bp, Cucumis melo
atgacggtcacaccgaaaatttctgtcaacgatggcaacttggtggttcacgggaagaccatactgactggggttcctgacaacattgtgctgaccccaggatctggccttggactcgttgctggcgctttcattggtgccactgcttcgaacagtaaaagtctacatgttttcccagtcggtgttttagagggtactcgcttcctatgttgtttccgtttcaagttatggtggatgacccaaagaatgggaacatctgggagagacatccctttcgagacacagttcctgctgatggagagcaagggtaacgatggagaggatcctgataattcttcgaccatctacaccgtcttccttcctctccttgagggccagttccgtgctgccctgcaaggaaatgaaaagaatgagatggagatttgcctcgagagtggagataacactgttgagaccaaccaaggactttctcttgtctatatgcatgctgggacaaatccctttgaagttatcactcaagcagtgaaggctgttgaaaagcatacgcaaacttttctacatagagagaagaaaaagttaccttccttccttgactggtttggttggtgtacttgggatgctttttacactgatgtcactgctgagggtgttgtggaaggtctcaaaagcctttcagagggaggggcacctccaaagttcttaatcatagatgatggttggcaacagatagaagccaaaccaaaagatgctgattgtgttgtacaagagggagcacagtttgcaagtaggctgtctggaataaaagaaaatcataagtttcagaaaaatgggaataactatgatcaggtcccaggcctaaaggtggttgttgatgatgccaagaaacaacacaaagtaaaatttgtgtatgcatggcatgctttggctggatattggggtggtgtgaaaccagcaagtccaggcatggagcattatgattccgctttggcgtacccggtccagtcaccgggtatgttgggcaaccaaccagacatagttgtagacagcttggctgttcatggcattggccttgtgcatccaaagaaagtctttaatttctataatgagcttcattcctacttggcttcctgtggtatcgatggcgtaaaggttgatgtgcaaaacattattgaaaccctcggtgctggtcatggtggcagggttacacttactcgtagctaccatcaggctcttgaagcttcgattgctcgtaacttttctgacaatggatgcattgcttgtatgtgccacaacactgacagtctctacagtgccaaacagactgcggtcgtgagagcttctgatgactattaccctcgtgatcctgcctcccacaccattcatatttcttctgtggcttacaattctcttttccttggagagttcatgcagcctgactgggatatgttccatagtttacatccgacagcagagtatcacggtgctgctcgtgcaattggcggatgtgcaatttatgtcagtgacaaaccaggtaaccacaactttgacctgttgaagaaactagtccttcccgatggatcagttcttcgtgctcagttacctggccgaccgacacgtgactctttgttcaacgatccagctagagatggcaccagcctgctcaaaatttggaatatgaacaaatgttctggtgttgttggagtattcaattgccaaggtgccggttggtgcaggatcacaaagaaaactcgcattcacgacgagtctccgggtacactcactacgtctgtccgtgcagctgatgttgatgctatttcgcaagttgcaggtgccgattggaagggtgatactattgtttatgcctatcgatcaggggatttgattcgattgccaaaaggtgcttcagttccagttaccctcaaagtcttggaatatgatcttctccatatttctcctctgaaggacatcgcatcgaacatctcatttgcaccaattggtctacttgacatgttcaacaccggtggtgctgtcgaacaagttaatgtccaagtggtcgaaccaataccagagttcgatggtgaagttgcttctgagctaacatgttctctccccaatgatcgacctccgacagctactatcaccatgaaagcccgaggatgcagaaggtttggtctatactcgtcccaacgtcctctgaaatgcagtgtggacaaggtcgatgtcgactttgtgtacgacgaggtcacagggttagtcaccttcgaaattcctatcccgacggaggaaatgtatagatggaacattgaaattcaagtt

SEQ ID NO: 4 - protein, 772 aa, Cucumis melo
MTVTPKISVNDGNLVVHGKTILTGVPDNIVLTPGSGLGLVAGAFIGATASNSKSLHVFPVGVLEGTRFLCCFRFKLWWMTQRMGTSGRDIPFETQFLLMESKGNDGEDPDNSSTIYTVFLPLLEGQFRAALQGNEKNEMEICLESGDNTVETNQGLSLVYMHAGTNPFEVITQAVKAVEKHTQTFLHREKKKLPSFLDWFGWCTWDAFYTDVTAEGVVEGLKSLSEGGAPPKFLIIDDGWQQIEAKPKDADCVVQEGAQFASRLSGIKENHKFQKNGNNYDQVPGLKVVVDDAKKQHKVKFVYAWHALAGYWGGVKPASPGMEHYDSALAYPVQSPGMLGNQPDIVVDSLAVHGIGLVHPKKVFNFYNELHSYLASCGIDGVKVDVQNIIETLGAGHGGRVTLTRSYHQALEASIARNFSDNGCIACMCHNTDSLYSAKQTAVVRASDDYYPRDPASHTIHISSVAYNSLFLGEFMQPDWDMFHSLHPTAEYHGAARAIGGCAIYVSDKPGNHNFDLLKKLVLPDGSVLRAQLPGRPTRDSLFNDPARDGTSLLKIWNMNKCSGVVGVFNCQGAGWCRITKKTRIHDESPGTLTTSVRAADVDAISQVAGADWKGDTIVYAYRSGDLIRLPKGASVPVTLKVLEYDLLHISPLKDIASNISFAPIGLLDMFNTGGAVEQVNVQVVEPIPEFDGEVASELTCSLPNDRPPTATITMKARGCRRFGLYSSQRPLKCSVDKVDVDFVYDEVTGLVTFEIPIPTEEMYRWNIEIQV

SEQ ID NO: 5 - protein, 767 aa, Tetragonia tetragonioides
MTITPSISVSNGNLVVHGKTILTGVPDNIILTPGSGAGLAAGAFIGATADDSKCLHVFPMGTLEGLRFMCCLRFKLWWMTQRMGKCGKDIPLETQFMIVESKDDTVEGEPDDSPTIYTVFLPLLEGQFRAVLQGTEKNEIEICLESGDTTVQTSQGLHLVYMHAGTNPYEVINQAVKAVEKHMQTFRHREKKRLPSFVDWFGWCTWDAFYTDVTAEGVDEGLRSLSEGGTPPRFLIIDDGWQQIGNEIVKDENCMVQEGAQFANRLTGIKENAKFQKKKNGEDKDQVPGLKHVVEEAKQRHNVKSVYVWHALAGYWGGVKPAAAGMEHYDTALAYPVQSPGVLGNQPDVVMDSLSVHGLGLVHPKKVFNFYNELHAYLAACGVDGVKVDVQNIIETLGAGHGGRVSLTRAYHQALEASIARNFPDNGCISCMCHNTDGIYSTKQTAVVRASDDFYPRDPASHTIHISSVAYNSLFLGEFMQPDWDMFHSLHPAADYHAAARAVGGCPIYVSDKPGFHNFELLKKLVLPDGSVLRARLPGRPTRDCLFNDPARDGTSLLKIWNKNNCSGVVGVFNCQGAGWCKIEKKIRIHDTSPGTLTGSVRATDVDSIAEVAGQGWNGDVVVYLYRAGELVCLPKGASLPVTLKVREYELFHFCPIKEITSNISFAPIGLLDMFNGSGAVDQFDVQLTSENRTELSDGEKRSPSASIQLKVRGCGRFGAYSSQCPLKCTVGGADSGFNYDEETCLLTLTLPVPQEEMYRWPVEIQV

SEQ ID NO: 6 - DNA, 2304 bp, Tetragonia tetragonioides
atgaccatcacaccaagcatttctgtcagtaacgggaaccttgtggttcatgggaagaccattttgactggagtgccagacaacatcatattgaccccaggctcaggtgcaggtcttgctgctggtgcatttatcggagctactgctgatgatagcaaatgtctccacgtattccccatgggcactttagagggtttgcgattcatgtgctgcctccgcttcaagctgtggtggatgactcaaaggatggggaaatgcggaaaagatattcccttagagacacagtttatgattgtggagagcaaggatgacactgtggaaggtgaaccagatgattctcccaccatctacactgtcttccttccccttttggagggccagtttcgtgctgttcttcaagggactgagaagaatgagatagagatttgcttggagagtggggacaccactgttcagaccagccaagggcttcatcttgtttacatgcatgctgggaccaacccctatgaagttatcaaccaggctgttaaggctgttgagaaacatatgcaaactttccgtcatcgagagaagaaaaggctgcctagttttgttgattggtttggctggtgcacatgggatgctttctacactgatgttacagctgagggtgtagacgaaggcctcagaagcttgtcagagggtggaactccaccgcggttcttgatcatagatgatggatggcagcagataggaaatgagattgttaaggatgaaaactgcatggtgcaagaaggcgctcaatttgccaatagactaactggaatcaaagagaatgccaagtttcaaaagaaaaagaatggtgaggacaaggatcaggttccaggcctaaagcatgttgtagaagaagccaaacaacgtcacaatgtgaagagtgtgtatgtgtggcatgctctggctggctattggggcggagtcaagccagcagctgcgggtatggaacactatgatacagcactggcatacccagttcagtctcctggcgtattgggtaaccagccagatgttgtcatggacagcctttctgttcatggtcttggattggtccatccgaagaaggtcttcaatttctacaatgagctccatgcctatctcgcggcatgtggagtagacggggtcaaagttgatgttcagaatataattgagacccttggtgccggccatggtggcagagtttccctcacccgtgcctatcaccaggctcttgaagcctctattgccaggaacttccctgataatggctgcatttcttgcatgtgtcacaacactgatggtatctatagtaccaagcagactgctgttgtgagggcttctgatgatttctatccacgtgatcctgcttcacacacgattcatatatcctctgtggcttataactcactcttccttggggagttcatgcaacctgattgggatatgttccatagcttgcacccagctgcagattatcatgcagcagctcgtgcagttggtggatgcccaatctatgtgagtgataaaccaggcttccacaactttgagctattgaagaagcttgtcctccctgatgggtcagtgctccgtgctcgtctgcctggaaggcctactcgtgattgcctctttaatgacccagccagagatgggaccagtttgctcaaaatctggaacaagaacaattgttctggtgtggttggtgtgttcaactgccaaggtgccggttggtgcaagattgagaaaaagattcggatccacgatacatctcctggaaccctcactggttctgtccgtgccactgacgtggattctattgccgaagttgctggtcaaggctggaatggggatgtggttgtgtatttgtaccgagcaggggaattggtttgcttacccaaaggagcttcacttccggtgactctcaaagtccgggaatatgaactcttccatttctgtcccatcaaggaaatcacaagcaacatttcatttgccccaattggcctgcttgatatgttcaatggcagtggagcagtagaccaatttgatgtgcaattaacatctgaaaatagaacagaactctccgatggtgagaagcgatccccaagcgcttccatccagctcaaagtgagaggatgtggccggtttggagcatactcttcccagtgccccctcaaatgtactgttggaggtgccgacagtggattcaactatgatgaagaaacctgcttgctaaccctcactctgcccgtcccccaagaggagatgtacagatggccagttgagattcaggtgtaa

SEQ ID NO: 7 - protein, 772 aa, Cucumis sativus
MTVTPKITVNDGNLVVHGKTILTGVPDNIVLTPGSGLGLVAGAFIGATASNSKSLHVFPVGVLEGTRFLCCFRFKLWWMTQRMGTSGRDIPFETQFLLMESQGNDGEDPDNSSTIYTVFLPLLEGQFRAALQGNEKNEMEICLESGDNTVETNQGLSLVYMHAGTNPFEVITQAVKAVEKHTQTFLHREKKKLPSFLDWFGWCTWDAFYTDVTAEGVVEGLQSLSDGGAPPKFLIIDDGWQQIEAKPKDADCVVQEGAQFASRLSGIKENHKFQKNGNNYDQVPGLKVVVDDAKKQHKVKFVYAWHALAGYWGGVKPASPGMEHYDSALAYPVQSPGMLGNQPDIVVDSLAVHGIGLVHPKKVFNFYNELHSYLASCGIDGVKVDVQNIIETLGAGHGGRVTLTRSYHQALEASIARNFSDNGCIACMCHNTDSLYSAKQTAVVRASDDYYPRDPASHTIHISSVAYNSLFLGEFMQPDWDMFHSLHPTAEYHGAARAIGGCAIYVSDKPGNHNFDLLKKLVLPDGSVLRAQLPGRPTRDSLFNDPARDGTSLLKIWNMNKCSGVVGVFNCQGAGWCRITKKTRIHDESPGTLTTSVRAADVDAISQVAGADWKGDTIVYAYRSGDLTRLPKGASVPVTLKVLEYDLFHISPLKDITSNISFAPIGLVDMFNIGGAVEQVDIQVVEPIPEFDGEVASELTCSLPDDRPPTATITMKARGCGRFGLYSSQRPLKCSVDKVGTDFVYDDVTGLVTFEIPIPTEEMYRWNIEIEV

SEQ ID NO: 8 - DNA, 2319 bp, Cucumis sativus
atgacggtcacaccgaagattactgtcaacgatggcaacttggtggttcacgggaagaccatactgactggggttcctgacaacattgtgctgaccccaggatctggccttggactcgttgctggcgctttcattggtgccactgcttcgaacagcaaaagtctacatgttttcccagtcggtgttttagagggtactcgcttcctttgttgtttccgtttcaagttatggtggatgactcaaagaatgggaacatctgggagagacatccctttcgagacacagttcctgctgatggagagccagggtaacgatggggaggatcctgataattcttcgaccatctacaccgtcttccttcctctccttgagggtcagttccgtgctgctctgcaaggaaatgaaaagaatgagatggagatttgcctcgagagtggagataacactgttgagaccaaccaaggactttctcttgtctatatgcatgctgggacaaatccctttgaagttatcactcaagcagtgaaggctgttgaaaagcatacgcaaacttttctacatagagagaagaaaaagttaccttccttccttgactggtttggttggtgtacttgggatgctttttacactgatgtcactgctgagggtgttgtggaaggcctccaaagcctttcagatggaggggcacctccaaaattcttaatcatagatgatggttggcaacagatagaagccaaaccaaaagatgctgattgtgttgtacaagagggagcacagtttgcaagtaggctgtctggaataaaagaaaatcataagtttcagaaaaatgggaataactatgatcaggtcccaggcctaaaggtggttgttgatgacgccaagaaacaacacaaagtgaaatttgtgtatgcatggcatgctttggctggttattggggtggagtgaaaccagcaagtccaggcatggagcattatgattctgctttggcgtacccggtccagtcgccgggtatgttgggcaaccaaccagacatagttgtagacagcttggctgttcatggcattggccttgtgcatccaaagaaagtctttaatttttataacgagctccattcctacttggcttcctgcggtattgacggcgtgaaggttgatgtgcaaaacattattgagaccctcggtgctggtcatggtggcagggttacacttactcgtagctaccatcaggctcttgaagcttcgattgctcgtaacttttctgacaatggatgcattgcttgtatgtgccacaacaccgacagtctctacagcgccaaacagactgcggtcgtgagagcttctgatgattattaccctcgtgatcctgcctcccacaccattcatatttcttctgtggcttacaattctcttttccttggagagttcatgcagcctgactgggatatgttccatagtttacatccgacagcagagtatcatggtgctgctcgtgcaattggcggatgtgcaatttatgtcagtgacaaaccaggcaaccacaactttgacctgttgaagaaactagtccttcccgatggatcagttcttcgtgcacagttacctggacgaccgacacgtgactctttgttcaacgatccagctagagatggcaccagcctgctcaaaatttggaatatgaacaaatgctctggtgttgttggagtattcaattgccaaggtgccggctggtgcaggatcacaaagaagactcgcattcacgatgagtctccgggtaccctaactacctctgtccgtgcagctgatgttgatgctatttcacaagttgcaggtgccgattggaagggtgatactatcgtttatgcctatcgatcaggagatttgactcgattgccaaaaggtgcttcagttcctgttaccctcaaagttttagaatacgatcttttccatatttctcctctgaaggacatcacctcaaacatctcgtttgccccaatcggtctagttgacatgttcaacatcggcggtgctgtcgagcaagtcgatatccaagtggtcgaaccaataccagagttcgatggtgaagttgcttctgagctaacatgttctctccctgatgatcgacctccaacggccactatcaccatgaaagcccgaggatgcggaaggttcggtttatactcgtcccaacggcctctgaaatgtagtgtggacaaggtgggtacggactttgtgtacgacgatgtgacagggttagtgaccttcgaaattcctatcccaacagaggagatgtatagatggaacattgaaattgaagtttag

SEQ ID NO: 9 - protein, 211 aa, Zea mays
PFEVITSSVKAVERHLQTFSHREKKKMPDILNWFGWCTWDAFYTNVTAQGVKQGLQSLEKGGVSPRFVIIDDGWQSVAMDPVGIACLSDNSANFANRLTHIRENHKFQKNGREGHREDDPAKGLAHVVNEIKGKHQLKYVYVWHAITGYWGGVRPGAAGMEHYGSKMQRPVPSPGVPKNERCEALDSMTANGLGLVNLDRAFSFYDELHSY

SEQ ID NO: 10 - DNA, 633 bp, Zea mays
ccgttcgaggtcatcacaagttcagtcaaggctgtcgagaggcacttgcagacgttctctcacagggagaagaaaaagatgccagacattctgaactggtttggctggtgcacgtgggacgcgttctacaccaatgtcaccgcccagggagtgaagcaaggattgcagagcttggaaaaaggcggggtctctcctaggttcgtcataatcgacgacggatggcagtccgtcgccatggaccctgtgggaatcgcttgcctatctgacaactcagccaacttcgcaaacaggctgactcacatcagggagaaccacaagtttcagaaaaatggcagggagggtcacagggaagatgacccagcgaagggcctagcacacgtcgtcaatgagattaaggggaagcatcagctcaagtatgtgtacgtatggcatgccatcaccggatactggggcggagtgaggccgggtgcagctggaatggagcactacggatcaaagatgcagcggcccgtgccatcgccgggggttccgaagaacgagcgctgcgaagccctggacagcatgacggccaacgggctgggcctcgtgaaccttgacagggcgttcagtttctacgacgagctccactcgtac

SEQ ID NO: 11 - protein, 747 aa, Zea mays
MTVASSVRLAGGNLTVCGRTVLSGVPDAVVATSAATEGAVDGIFLGADFAEPAARHVVSLGDLRDVRFMACFRFKLWWMAQRMGEKGSDVPRETQFLLVESRGVGDEDAAYVVFLPLVEGAFRASIQGGAGDALELCVESGDDDTRAASFERSLFVGAAESDPFAAISGAVAAAKSALRTFRVRAEKKLPGIVDYFGWCTWDAFYQDVTQEGVEAGLRSLIAGGAPPKFVIIDDGWQSVATDTNESAGEDKPPLLSRLTGIKENSKFQNADDPAAGIKTVVRLAKEEYRLKYVYVWHAITGYWGGVRPGEEHYRSSMQFPKVSPGVMENEPGMKTDVLTVQGLGLVHPRAVYRFYDELHAYLAAAGVDGVKVDVQCILETLGAGHGGRVQLTRQYHQALDASVAKNFPENGIIACMSHNTDALYCSKQTAVVRASDDFCPRDPASHTIHIASVAYNSVFLGEFMLPDWDMFHSLHQAGDYHGSARAISGGPVYVSDAPGKHNFELLKKIVLPDGSILRARLPGRPTKDCLFTDPARDGVSLLKIWNMNKFTGVLGVYNCQGAAWNSVEKKNTFHQTGTEALTCGVKGGDVHLISEAATDTEWDGDCAMYRHADGDLVVLPHDAALPVSLKVLEHDILTVSPIKELAPGFRFAPIGLVDMFNSGGAVEGLTYHLLGGDGSTLGSEAVALACMEVKGCGRFGAYSSVRPRKSTLGSAQIELKYDSSSGLLILQLDAMPKERVHKIVIEL

SEQ ID NO: 12 - DNA, 2244 bp, Zea mays
atgacggtcgcctcgtccgtcaggctcgccggcggcaatctgacggtatgcgggcggacggtgctgtccggggtgccggacgcggtggtggccacgtcggcggcgacggagggagcggtcgacgggatcttcctcggcgccgacttcgccgagccggccgcccggcacgtcgtctccctcggcgacctgagggacgttcggttcatggcgtgcttccgattcaagctatggtggatggcgcagcggatgggggagaaaggcagcgacgtcccgcgcgagacccagttcctgctcgtcgagtccaggggcgtcggcgacgaggacgcggcgtacgtcgtgttcctcccgctcgtggagggcgcgttccgggccagcatccaaggcggtgccggcgacgcgctggagctctgcgtcgagagcggggacgacgacacgcgcgcggcgtccttcgagcgatccctcttcgtgggcgccgcggagtccgaccccttcgcggccatctccggcgcagtcgccgccgccaagtccgcgctcaggacgttccgggtccgcgccgagaagaagctcccgggcatcgtcgactacttcggctggtgcacctgggacgccttttaccaggacgtcacccaggagggcgtcgaggccgggctccgcagcctcatcgccggcggcgcgccgcccaagttcgtcatcatcgacgacggctggcagtccgtcgccaccgacaccaacgaatcggccggagaggacaagccgcccctcctatctcggctcaccggcatcaaggagaacagcaagttccagaacgcggacgacccggccgccggcatcaagacggtggtgcgcttggcgaaggaggagtaccggctcaagtacgtctacgtgtggcacgccatcaccggctactggggcggcgtccggcccggcgaggagcactaccgctccagcatgcagttccccaaggtttcgccgggcgtcatggagaacgagcccggcatgaagaccgacgtgctcaccgtgcaggggctcggcctcgtgcacccgcgcgccgtgtaccgcttctacgacgagctccacgcgtacctcgccgccgccggagtcgacggcgtcaaggtggacgtgcagtgcatcctggagacgctcggcgccggccacggcggccgcgtgcagctcaccaggcagtaccaccaggcgctggacgcctccgtcgccaagaacttcccggagaacggcatcatcgcctgcatgagccacaacaccgacgccctttactgctccaagcagacggcggtggtgagggcgtcggatgatttctgcccaagggaccccgcgtcgcacacgatccatatcgcctcggtggcgtacaacagcgtgttcctcggcgagttcatgctcccggactgggacatgttccactcgctccaccaggccggcgactaccacggctccgcccgcgccatcagcggaggccctgtctacgtcagtgacgcacccggcaagcacaacttcgagctgctgaagaagattgtcttgcccgacggttccattctccgcgcgcgtctgcctggccggccgaccaaggattgcctgttcacggacccggcacgggacggcgtaagcctgctcaagatctggaacatgaacaagttcaccggcgtgctgggcgtgtacaactgccagggcgcggcgtggaactccgtggagaagaagaacaccttccaccagaccggcaccgaggccctgacctgcggcgtcaagggcggcgacgtccacctcatctccgaggccgcgacggacaccgaatgggacggcgactgcgccatgtaccggcatgccgacggcgacctcgtcgtcctcccgcacgacgcggcgttgcccgtctccctcaaggtcctggaacatgacatcctcaccgtgtcacccatcaaggagttggcgcctggtttcaggttcgccccgatcgggctggtggacatgttcaatagcggcggggcggtggaaggcctgacctatcacctcctcggcggcgacggttccaccttgggctccgaggctgtcgcattggcgtgcatggaggtgaagggctgtggaaggttcggtgcctactcttcggtcaggccaaggaagtccacgctcggttcagctcagattgagctcaagtatgattcttcctcagggctgctgattctgcagctggatgcgatgcccaaggagagggttcacaagattgttattgagttgtag

SEQ ID NO: 13 - protein, 753 aa, Oryza sativa
MTVGAGVAVQDGGLVALGATVLTEVRDNVLLTPAAGAGMTSGTFVGVRSATAGSRSVFPVGKLRGLRFICTFRFKMWWMTQRMGTSGRDIPFETQFLLVEAADADGSHLAGDGAAAVYTVFLPILEGPFRAVLQGNSDDELEICLESGDPAVESFEGTHLVFVGAGSDPFEVITNSVKAVERHLQTFTHREKKKMPDMLNWFGWCTWDAFYTDVTSEGVMEGLQSLGKGGTGPKFVIIDDGWQSVSMDPAGIASLADNSANFANRLTHIKENHKFQLNGRKGHREENPANGLAHIVNEIKGKHQLKYVYVWHAITGYWGGVRPGADGMEHYESKMQYPVSSPGVQKNEPCDALNSITTNGLGLVNPDRVFSFYNELHAYLASAGIDGVKVDVQNILETLGAGHGGRVLLARKYHQALEASIARNFRDNGIICCMSHNTDNLYSSKRSAVVRASDDFWPRDPASHTIHIASVAYNTVFLGEFMQPDWDMFHSVHPMAEYHAAARAVGGCAIYVSDKPGNHDFNLLKKLVLPDGSILRAKLPGRPTRDCLFSDPARDGKSILKIWNLNEHSGVIGAFNCQGAGWCRVGKKNLVHDEQPATVTGVIRAQDVHHLATVAADGWNGDVIVYSHIGGEVTCLPKNASLPVTLKTREYEVFTVVPLKKLDNGVSFAAVGLIGMFNSGGAVTAVRYVEDAGVEVRVRGSGTVGAYSSAKPARVVVDSEAAEFSYDDGCGLVTFELAVPEQELYSWTISIEY

SEQ ID NO: 14 - DNA, 2262 bp, Oryza sativa
atgacggtgggagccggggtggcggtgcaggacggcggcctggtggcgctgggcgccacggtgctgacggaggtgcgcgacaatgtgctcctgacgccggccgccggcgccggcatgacgagcggcacgttcgtcggagtccgctccgccaccgccggcagccgcagcgtcttccccgtcgggaagctcaggggattgcggttcatctgcacgttccggtttaagatgtggtggatgacgcagaggatggggacgtcaggccgcgacatccccttcgagacgcagttcctcctcgtcgaggccgccgacgccgacggatcacacctcgccggcgacggcgccgccgcggtgtacaccgtgttcctcccgatcttggagggaccgttccgagctgtgcttcaggggaactctgatgatgagctcgagatttgcctcgagagtggtgacccagctgtggaatcattcgaaggcacgcatctggttttcgtcggtgccggatcggatccattcgaagtcatcacaaattctgtcaaggctgttgagaggcacttgcagacgtttactcacagggaaaagaagaagatgccagacatgctaaactggtttggttggtgcacatgggatgcgttttatactgatgttacttcagaaggagtaatggaaggattacagagtctaggaaaaggtggaactggcccaaaatttgtgataattgatgatggatggcaatcagttagtatggatcctgcaggaattgcatcattagctgataactcagccaactttgcaaacaggttgactcacataaaggagaaccacaaatttcagctaaacgggaggaaaggtcacagggaagagaatccggcaaatggccttgcacacattgtcaatgaaattaagggcaaacatcagctgaagtatgtttatgtgtggcacgcgatcaccggatactggggtggagtaaggcctggtgctgatggaatggagcactacgaatcgaagatgcagtacccggtctcatcaccgggagttcagaagaacgagccatgtgatgctctgaacagcataaccaccaacggccttggccttgtgaaccctgacagagtgttcagcttctacaacgagctacacgcctaccttgcatctgctgggatcgatggagtgaaagtagatgtgcagaacattcttgagacactgggtgctggccatggtggaagagtgcttctggcaaggaagtatcaccaggctctagaagcttccatcgcccggaacttccgcgacaacggcatcatatgctgcatgagccacaacacggataacttgtacagttctaagaggagtgctgttgtgagagcttctgatgatttctggcctagagaccctgcttcccatactatacatattgcatctgttgcatataatactgtatttcttggagaattcatgcaaccagattgggacatgttccatagtgttcacccaatggctgaataccatgctgcagcaagagcagtcggtggctgtgccatatatgtcagtgacaagcctgggaaccatgacttcaatttgctgaagaagctggttcttcctgacggatcgatcctgagagccaaactccccggcaggccaaccagagactgtctgttttcagaccctgcaagggatggcaagagtatcctgaagatatggaatctgaacgagcactccggtgtgattggcgccttcaattgccagggtgctggatggtgcagagtagggaagaagaacctcgtccacgacgagcagccggcgacggtcaccggtgtcatccgtgcacaggacgtgcatcacctcgcaaccgttgctgccgatggctggaacggcgacgtgatcgtctactcgcacatcggaggggaggtgacctgcctgcccaagaacgcgtcgttgccggtgacactgaagacgcgagagtacgaggtcttcaccgtcgtaccactgaagaagctcgacaatggcgtctccttcgccgcggtcgggctgatcgggatgttcaactccggcggggcggtgacggcggtgaggtacgtggaggacgccggcgttgaggtcagggtgcgtggctccggcacggtcggggcgtactcctcggcgaagccggcgagggtggtggtggattcggaggcggcggaattctcctacgatgatggatgcggcttggtcacgttcgagctcgccgtgccggagcaagaactctactcctggaccatctcgatcgagtactga

SEQ ID NO: 15 - protein, 777 aa, Pisum sativum
MTVTPKISVNDGNLVVHGKTILKGVPENVVLTPGSGNGLLTGGAFIGATASNSKSLHVFPIGILEGLRFVCCFRFKLWWMTQRMGTCGRDIPLETQFMLIESKDSEGEEGNSPVIYTVLLPLLEGPFRSVLQGNEKSEIEICFESGDHAVETNQGLHMVYMHAGTNPFEVINQAVKAVEKHMQTFHHREKKRLPSFLDMFGWCTWDAFYTDVTAEGVEQGLKSLSEGGTPPRFLIIDDGWQQIESKAKDPGCVVQEGAQFATMLTGIKENAKFQKNKNEEHSEPTSGLKHLVDGVKKHHNVKNVYVWHALAGYWGGVKPAATGMEHYDTALAYPVQSPGVLGNQPDIVMDSLSVHGLGLVHPKKVFNFYNELHAYLASCGVDGVKVDVQNIIETLGAGHGGRVSLTRSYHHALEASIARNFSDNGCIACMCHNTDGLYSAKQTAVVRASDDFYPRDPASHTIHISSVAYNSLFLGEFMQPDWDMFHSLHPAAEYHAAARAIGGCPIYVSDKPGNHNFDLLKKLVLSDGSVLRAQLPGRPTRDSLFVDPARDRTSLLKIWNMNKCTGVVGVFNCQGAGWCKVEKKTRIHDISPGTLTSSVCASDVDLITQVAGAEWHGETIVYAYRSGEVIRLPKGVSIPVTLKVLEFELFHFCPIQEISSSISFATIGLMDMFNTGGAVEEVEIHRETDNKQELFEGEAVSSELITSLGPNRTTTATITLKVRGSGKFGVYSSQRPIKCMVDGTETDFNYDSETGLTTFIIPVPQEELYKWLIEIQV

SEQ ID NO: 16 - DNA, 2334 bp, Pisum sativum
atgactgtgactccgaagatttctgttaacgatggaaacttggttgttcatggcaagactatacttaaaggagttccagaaaatgttgtgcttactccaggttctggcaacggtcttcttaccggcggtgcgttcatcggtgcaactgcttccaattccaaaagcctacatgttttcccgatcggaatcttagaggggcttcggtttgtgtgttgcttccggttcaagttatggtggatgactcagcgaatgggaacttgtgggagggatattcctcttgagactcagtttatgcttatagagagcaaagacagtgaaggggaagagggaaattctccggttatttacactgttttgcttcctctattggaaggcccgtttcgatctgttctacaaggaaatgagaaaagcgagatcgagatttgctttgagagtggtgatcatgctgttgagactaatcaaggccttcacatggtttacatgcatgctggcaccaacccttttgaagtcatcaaccaagctgtcaaggctgtggaaaagcacatgcaaacatttcatcatcgtgagaagaaaaggctgccatcttttcttgacatgtttggttggtgcacatgggatgctttctatactgatgtaacagctgagggcgttgaacaaggcctgaaaagtctatcagagggaggtacacctccgcggtttctcatcatagatgatggctggcaacagattgaaagtaaagcaaaggatcctggctgtgttgtgcaagaaggagcacagtttgctactatgttgactggtattaaagagaatgcaaaatttcaaaagaataaaaatgaagagcacagcgaaccgacatcgggtcttaaacatcttgttgacggagtgaagaaacatcacaatgtcaaaaatgtttatgtatggcatgcactagctggttattggggtggagtgaagccagcagcaactggcatggaacattatgacacggctttggcatacccggtgcaatcgcccggagtattaggaaaccaaccagacattgtcatggacagcttgtctgtacatggcctcggtctagtacatccaaagaaggttttcaacttctacaatgagctccatgcttatttagcttcatgtggagtagacggagtcaaggtggacgtgcagaacattattgaaacccttggcgcaggacacggtggccgagtctcacttacccgcagctatcatcatgcactcgaggcttccattgctcgtaacttttccgacaatggatgcatagcgtgtatgtgtcataacactgatggactttatagtgctaagcagactgctgttgtgagagcctctgatgatttttacccacgtgatcctgcttcccacacgatccatatttcgtctgttgcatacaattcacttttccttggagagtttatgcaaccagattgggacatgttccacagtttacatccagcagctgagtatcatgctgcagctcgtgcaatcggtggatgtccaatttatgtcagtgataagcctggcaaccacaattttgatcttcttaagaaattggttctttctgatggttcagttctccgcgctcagttgcctggcagacctacacgtgactctctatttgttgatccggcgagagataggactagcctgctcaaaatatggaacatgaacaaatgtactggagttgttggtgtatttaactgccaaggtgctgggtggtgcaaggtagagaagaaaacccgcatccatgatatatctcctggcacacttactagctctgtttgtgcctctgatgttgatctcatcacccaagtagctggtgctgaatggcatggggagactattgtttatgcttacagatcaggcgaggtgatccggctaccgaaaggtgtttcaattccagtgacactaaaggttctggagtttgaacttttccatttctgtccaatccaagaaatttcatccagtatatcatttgcaacaataggcctaatggatatgttcaacactggaggagctgtggaagaggtcgaaattcatagagagactgacaacaaacaagaactatttgaaggagaggctgtatcgtcggaactgataacttctctaggtcctaaccgaacgacaacagcaactattactttgaaagttagaggaagtggaaagtttggagtttactcctcgcagcgtccaataaagtgcatggtggatgggactgaaactgacttcaactatgactcagaaactggattgacaaccttcatcatcccagttcctcaagaggagttgtataaatggttaattgagattcaagtttaa

SEQ ID NO: 17 - protein, 753 aa, Cucumis sativus
MTVGAGITISDANLTVLGNRVLSDVHNNITLTAAPGGGVMNGAFIGVQSDQIGSRRVFPIGKLIGLRFLCAFRFKLWWMTQRMGCSGQEIPFETQFLVVETRDGSNIAGNGEEGDAVYTVFLPILEGDFRAVLQGNDNNELEICLESGDPSVDGFEGSHLVFVGAGSDPFETITYAVKSVEKHLQTFAHRERKKMPDILNWFGWCTWDAFYTDVTSDGVKKGLESFENGGIPPKFVIIDDGWQSVAKDAASTDCKADNTANFANRLTHIKENYKFQKDGKEGERIENPALGLQHIVSYMKEKHATKYVYVWHAITGYWGGVSSGVKEMEQYESKIAYPVASPGVESNEPCDALNSISKTGLGLVNPEKVFNFYNEQHSYLASAGVDGVKVDVQNILETLGAGHGGRVKLARKYHQALEASISRNFQDNGIISCMSHNTDGLYSSKRNAVIRASDDFWPRDPASHTIHIASVAYNSLFLGEFMQPDWDMFHSLHPMAEYHGAARAVGGCAIYVSDKPGQHDFNLLKKLVLHDGSILRAKLPGRPTKDCLFADPARDGKSLLKIWNMNDLSGVVGVFNCQGAGWCKVGKKNLIHDENPDTITGVIRAKDVSYLWKIAGESWTGDAVIFSHLAGEVVYLPQDASMPITLKSREFDVFTVVPVKELANDIKFAPIGLMKMFNSGGAVKEMNHQPGSSNVSLKVRGSGPFGAYSSSKPKRVAVDSEEVEFIYDEGGLITIDLKVPEKELYLWDIRIEL

SEQ ID NO: 18 - DNA, 2262 bp, Cucumis sativus
atgacggttggtgctggaattactatatccgacgccaatttgacggtgttgggaaatcgtgttttatctgatgttcataataacattactctcacggcagcgccgggaggtggtgtgatgaacggcgccttcataggagttcaatctgatcagatcggtagtcgccgagtttttcctattgggaaattgatagggttgaggttcttatgtgcttttcgattcaaattatggtggatgactcaaagaatggggtgttctggtcaagaaattccattcgagacacaatttcttgtggtggaaacacgtgatggttctaacattgccggaaatggagaggaaggcgatgccgtttatactgtttttcttcctattcttgaaggcgatttcagagctgttcttcaagggaatgataacaatgaacttgaaatctgtttagaaagcggagatccaagtgtagatgggtttgagggtagccatttggtgtttgtgggtgctggatcagatccttttgaaaccattacttatgcagtcaagtccgttgagaagcatttgcaaacttttgctcatcgtgaaagaaaaaagatgcctgatattttgaactggttcggctggtgcacctgggatgctttctacactgatgtcacttcagatggcgtcaagaagggtcttgaaagctttgagaatggaggaattcctcccaagtttgtcattatcgatgatggatggcaatcagttgctaaggatgctgctagtactgattgcaaagctgataacacagcaaactttgcaaacaggttaacacacataaaagagaattacaaatttcaaaaagatggcaaagagggtgaaagaattgagaaccctgcacttggtcttcaacacattgtgtcctacatgaaagagaagcatgcgacgaagtatgtttatgtttggcatgccataacaggctactggggtggtgtgagttctggagttaaagagatggaacaatatgagtccaagattgcgtacccggttgcatctcctggggtcgaatcaaacgagccatgtgatgctttgaatagcatcagcaaaactggacttggcctagtgaaccctgaaaaagttttcaacttttacaatgaacaacactcgtatcttgcatctgctggtgtcgatggagttaaagttgatgttcaaaacattcttgagacgcttggagcaggtcatggtggaagagttaaacttgctagaaaataccatcaggctcttgaggcatcaatttctcgaaattttcaagataacggaatcatttcgtgtatgagtcataataccgacggtttatacagttcaaagaggaatgctgttattcgagcttccgacgatttttggcctagagatccagcatctcacacgattcatatagcatcagttgcttacaactccttatttcttggggagtttatgcagccagattgggatatgtttcatagtcttcatcctatggccgaatatcatggagcagctcgtgccgtgggaggatgtgctatatatgtcagtgacaagcctggtcaacatgacttcaatcttttgaagaagcttgtgctccatgatggttctattctgagagctaagctccccggacgaccgacaaaggactgcctatttgcggatcctgctagagatggaaaaagtctattgaagatttggaatatgaatgatctttctggagttgttggggtctttaactgccaaggagcaggatggtgtaaggttggaaagaaaaacctcattcatgacgagaatcccgacacaatcacaggggttattcgagctaaggatgttagttatctatggaagattgcaggcgagtcctggacaggggatgcagtgatattctcccatcttgctggagaagttgtttacctaccacaagatgcatcgatgccaataaccttgaaatctcgagagttcgatgtcttcacagttgttcctgtcaaggaactagctaatgacatcaaatttgctcctataggtttgatgaagatgttcaactctggaggagctgtgaaagaaatgaaccatcaacctggaagttcaaacgtgtcactgaaagttcggggttccgggccattcggggcatattcctcgagcaaaccgaagcgtgtagcagtggactcggaggaggtagagttcatatatgatgagggtggtttaatcaccattgacttgaaggtaccagagaaagagttgtatctttgggacataagaattgaactatga

SEQ ID NO: 19 - protein, 772 aa, Cucumis melo
MTVTPKISVNDGNLVVHGKTILTGVPDNIVLTPGSGLGLVAGAFIGATASNSKSLHVFPVGVLEGTRFLCCFRFKLWWMTQRMGTSGRDIPFETQFLLMESKGNDGEDPDNSSTIYTVFLPLLEGQFRAALQGNEKNEMEICLESGDNTVETNQGLSLVYMHAGTNPFEVITQAVKAVEKHTQTFLHREKKKLPSFLDWFGWCTWDAFYTDATAEGVVEGLKSLSEGGAPPKFLIIDDGWQQIEAKPKDADCVVQEGAQFASRLSGIKENHKFQKNGNNYDQVPGLKVVVDDAKKQHKVKFVYAWHALAGYWGGVKPASPGMEHYDSALAYPVQSPGMLGNQPDIVVDSLAVHGIGLVHPKKVFNFYNELHSYLASCGIDGVKVDVQNIIETLGAGHGGRVTLTRSYHQALEASIARNFSDNGCIACMCHNTDSLYSAKQTAVVRASDDYYPRDPTSHTIHISSVAYNSLFLGEFMQPDWDMFHSLHPTAEYHGAARAIGGCAIYVSDKPGNHNFDLLKKLVLPDGSVLRAQLPGRPTRDSLFNDPARDGISLLKIWNMNKCSGVVGVFNCQGAGWCRITKKTRIHDESPGTLTTSVRAADVDAISQVAGADWKGDTIVYAYRSGDLIRLPKGASVPVTLKVLEYDLLHISPLKDIASNISFAPIGLLDMFNTGGAVEQVNVQVVEPIPEFDGEVASELTCSLPNDRPPTATITMKARGCRRFGLYSSQRPLKCSVDKVDVDFVYDEVTGLVTFEIPIPTEEMYRWDIEIQV

SEQ ID NO: 20 - DNA, 2319 bp, Cucumis melo
atgacggtcacaccgaaaatttctgtcaacgatggcaacttggtggttcacgggaagaccatactgactggggttcctgacaacattgtgctgaccccaggatctggccttggactcgttgctggcgctttcattggtgccactgcttcgaacagtaaaagtctacatgttttcccagtcggtgttttagagggtactcgcttcctatgttgtttccgtttcaagttatggtggatgacccaaagaatgggaacatctgggagagacatccctttcgagacacagttcctgctgatggagagcaagggtaacgatggagaggatcctgataattcttcgaccatctacaccgtcttccttcctctccttgagggccagttccgtgctgccctgcaaggaaatgaaaagaatgagatggagatttgcctcgagagtggagataacactgttgagaccaaccaaggactttctcttgtctatatgcatgctgggacaaatccctttgaagttatcactcaagcagtgaaggctgttgaaaagcatacgcaaacttttctacatagagagaagaaaaagttaccttccttccttgactggtttggttggtgtacttgggatgctttttacactgatgccactgctgagggtgttgtggaaggtctcaaaagcctttcagagggaggggcacctccaaagttcttaatcatagatgatggttggcaacagatagaagccaaaccaaaagatgctgattgtgttgtacaagagggagcacagtttgcaagtaggctgtctggaataaaagaaaatcataagtttcagaaaaatgggaataactatgatcaggtcccaggcctaaaggtggttgttgatgatgccaagaaacaacacaaagtaaaatttgtgtatgcatggcatgctttggctggatattggggtggtgtgaaaccggcaagtccaggcatggagcattatgattccgctttggcgtacccggtccagtcaccgggtatgttgggcaaccaaccagacatagttgtagacagcttggctgttcatggcattggccttgtgcatccaaagaaagtctttaatttctataatgagcttcattcctacttggcttcctgtggtatcgatggcgtaaaggttgatgtgcaaaacattattgaaaccctcggtgctggtcatggtggcagggttacacttactcgtagctaccatcaggctcttgaagcttcgattgctcgtaacttttctgacaatggatgcattgcttgtatgtgccacaacactgacagtctctacagtgccaaacagactgcggtcgtgagagcttctgatgactattaccctcgtgatcctacctcccacaccattcatatttcttctgtggcttacaattctcttttccttggagagttcatgcagcctgactgggatatgttccatagtttacatccgacagcagagtatcacggtgctgctcgtgcaattggcggatgtgcaatttatgtcagtgacaaaccaggtaaccacaactttgacctgttgaagaaactagtccttcccgatggatcagttcttcgtgctcagttacctggccgaccgacacgtgactctttgttcaacgatccagctagagatggcatcagcctgctcaaaatttggaatatgaacaaatgttctggtgttgttggagtattcaattgccaaggtgccggttggtgcaggatcacaaagaaaactcgcattcacgacgagtctccgggtacactcactacgtctgtccgtgcagctgatgttgatgctatttcgcaagttgcaggtgccgattggaagggtgatactattgtttatgcctatcgatcaggggatttgattcgattgccaaaaggtgcttcagttccagttaccctcaaagtcttggaatatgatcttctccatatttctcctctgaaggacatcgcatcgaacatctcatttgcaccaattggtctacttgacatgttcaacaccggtggtgctgtcgaacaagttaatgtccaagtggtcgaaccaataccagagttcgatggtgaagttgcttctgagctaacatgttctctccccaatgatcgacctccgacagctactatcaccatgaaagcccgaggatgcagaaggtttggtctatactcgtcccaacgtcctctgaaatgcagtgtggacaaggtcgatgtcgactttgtgtacgacgaggtcacagggttagtcaccttcgaaattcctatcccgacggaggaaatgtatagatgggacattgaaattcaagtttaa

SEQ ID NO: 21 - protein, 378 aa, Coffea arabica
MVKSPGTEDYTRRSLLANGLGLTPPMGWNSWNHFRCNLDEKLIRETADAMVSKGLAALGYKYINLDDCWAELNRDSQGNLVPKGSTFPSGIKALADYVHSKGLKLGIYSDAGTQTCSKTMPGSLGHEEQDAKTFASWGVDYLKYDNCNNNNISPKERYPIMSKALLNSGRSIFFSLCEWGEEDPATWAKEVGNSWRTTGDIDDSWSSMTSRADMNDKWASYAGPGGWNDPDMLEVGNGGMTTTEYRSHFSIWALAKAPLLIGCDIRSMDGATFQLLSNAEVIAVNQDKLGVQGNKVKTYGDLEVWAGPLSGKRVAVALWNRGSSTATITAYWSDVGLPSTAVVNARDLWAHSTEKSVKGQISAAVDAHDSKMYVLTPQ